(19) United States
(12) Patent Application Publication
(10) Pub. No.: US 2018/0177833 A1
(43) Pub. Date: Jun. 28, 2018

(54) BIFIDOBACTERIA AS PROBIOTIC FOUNDATION SPECIES OF GUT MICROBIOTA

(71) Applicant: PERFECT (CHINA) CO., LTD., Zhongshan, Guangdong (CN)

(72) Inventors: Liping ZHAO, Shanghai (CN); Chenhong ZHANG, Shanghai (CN); Huan WU, Shanghai (CN); Guojun WU, Shanghai (CN)

(73) Assignee: PERFECT (CHINA) CO., LTD., Zhongshan, Guangdong (CN)

(21) Appl. No.: 15/738,122

(22) PCT Filed: Jun. 30, 2015
(86) PCT No.: PCT/CN2015/082887

§ 371 Date: Dec. 20, 2017

(51) Int. Cl. A61K 35/745; A61K 35/747; A61P 3/10; A61P 43/00

(52) U.S. Cl. CPC A61K 35/745; A61P 43/00; A61P 3/10; A61K 35/747

(57) ABSTRACT

This invention relates to novel probiotic Bifidobacteria strains, particularly, a B. pseudocatenulatum strain, and its use as probiotic, and food products, feed products, dietary supplements and pharmaceutical formulations containing them. The bacteria are suitable for the treatment of obesity, diabetes (particularly Type 2 diabetes), and related conditions.

### FIELD

This invention relates to novel Bifidobacteria strains and their uses, to food products, feed products, dietary supplements and pharmaceutical formulations containing them, and to methods of making and using these compositions.

### BACKGROUND

Probiotics, generally understood to mean “live microorganisms that when administered in adequate amounts confer a health benefit on the host,” have been used widely for the prevention and treatment of a wide range of diseases, and there is strong evidence for their efficacy in some clinical scenarios. For example, WO 2007/043933 describes the use of probiotic bacteria for the manufacture of food and feed products, dietary supplements, for controlling weight gain, preventing obesity, increasing satiety, prolonging satiation, reducing food intake, reducing fat deposition, improving energy metabolism, enhancing insulin sensitivity, treating obesity and treating insulin insensitivity.

WO 2009/024429 describes the use of a primary composition comprising an agent that reduces the amount of proteobacteria, in particular enterobacteria and/or deferribacteres in the gut for the treatment or prevention of metabolic disorders, to support and/or to support weight management.

WO 2009/004076 describes the use of probiotic bacteria for normalising plasma glucose concentrations, improving insulin sensitivity, and reducing the risk of development in pregnant women, and preventing gestational diabetes.

WO 2009/021824 describes the use of probiotic bacteria, in particular Lactobacillus rhamnosus, to treat obesity, treat metabolic disorders, and support weight loss and/or weight maintenance.

WO 2008/016214 describes a probiotic lactic acid bacterium of the strain Lactobacillus gasseri BNR17 and its use in the inhibition of weight gain.

WO 02/38165 describes use of a strain of Lactobacillus (in particular, Lactobacillus plantarum) in reducing the risk factors involved in the metabolic syndrome.

US 2002/0037577 describes the use of microorganisms, such as Lactobacilli, for the treatment or prevention of obesity or diabetes mellitus by reduction of the amount of monosaccharide or disaccharide which may be absorbed into the body, by converting such compounds into polymeric materials which cannot be absorbed by the intestine.

Lee et al., J. Appl. Microbiol. 2007, 103, 1140-1146, describes the anti-obesity activity of trans-10, cis-12-conjugated linoleic acid (CLA)-producing bacterium of the strain Lactobacillus plantarum PL62 in mice.

Li et al., Hepatology, 2003, 37(2), 343-350, describe the use of probiotics and anti-TNF antibodies in a mouse model for non-alcoholic fatty liver disease.

US2014/0369965 discloses a Bifidobacterium pseudocatenulatum strain isolated from the feces of healthy breastfeeding mice. The same document further discloses the use of this strain, along with its cell components, metabolites, and secreted molecules, and combinations thereof with other microorganisms for the prevention and/or treatment of obesity, overweight, hyperglycemia and diabetes, hepatic steatosis or fatty liver, dyslipidemia, metabolic syndrome, immune system dysfunction associated with obesity and overweight; and an unbalanced composition of the intestinal microbiota associated with obesity and overweight. However, this strain is not derived from humans.

In other words, existing probiotics have many limitations, and there is a need for new strains of probiotic microorganisms.

### SUMMARY

In one aspect, the invention discloses the use of a bacterium of the genus Bifidobacterium or a mixture thereof in the manufacture of a food product, dietary supplement or medicament for treating obesity, controlling weight gain and/or inducing weight loss in a mammal.

In another aspect, the invention discloses a composition comprising (1) a Bifidobactgerium pseudocatenulatem strain C95 with accession No. CGMCC10549, wherein the genome of the C95 strain is designated as a reference genome; (2) a highly similar strain, wherein the highly similar strain comprises a genome that is designated as a query genome, wherein when aligned, the query genome covers at least 86% of the reference genome, the query and reference genomes share at least 98.7% sequence identity in aligned regions; or (3) a strain derived therefrom; (4) a pharmaceutically acceptable or dietary carrier.

In another aspect, the invention discloses a method for preparing the composition of the present invention, comprising formulating the Bifidobactgerium pseudocatenulatem strain C95 or the highly similar strain into a suitable composition.

In another aspect, the invention discloses a method for the prevention and/or treatment of a disease selected from the group consisting of overweight, obesity, hyperglycemia, diabetes, fatty liver, dyslipidemia, metabolic syndrome, infections in obese or overweight subjects and/or adipocyte hypertrophy said method comprising the administration of the composition of the present invention to a subject in need thereof.

In another aspect, the invention discloses a method for reducing simple or genetic obesity, alleviating metabolic deteriorations, or reducing inflammation and fat accumulation in a subject in need thereof, comprising the administration of the composition of the present invention to a subject in need thereof.

In another aspect, the invention discloses a method for establishing as foundation species that define the structure of a healthy gut ecosystem, rendering a gut environment unfavorable to pathogenic and detrimental bacteria, reducing the concentration of enterobacteria in intestinal content with respect to an untreated control, the method comprising the administration of the composition of the present invention to a subject in need thereof.

In another aspect, the invention discloses a method for treating diabetes in a subject in need thereof, comprising the administration of the composition of the present invention to a subject in need thereof.

### BRIEF DESCRIPTION OF THE DRAWINGS

FIG. 1A illustrates that after 30 days of intervention, the SO cohort lost 9.5±0.4% (mean±s.e.m.) of their initial bodyweight, and the PWS cohort lost 7.6±0.6%.

FIG. 1B illustrates that aspartate aminotransferase (AST) and alanine aminotransferase (ALT) levels in the blood were reduced, indicating improved liver condition.

FIG. 1C illustrates that glucose homeostasis was improved, indicating better insulin sensitivity.

FIG. 1D illustrates that blood levels of total cholesterol, triglycerides, and low-density lipoprotein (LDL) were decreased.

FIG. 1E illustrates that several markers of systemic inflammation were also improved in PWS and SO cohorts after 30 days of dietary intervention, including C-reactive protein (CRP), serum amyloid A protein (SAA), α-acid glycoprotein (AGP) and white blood cell count (WBC).

FIG. 2A illustrates that mice maintained weight for 4 days after transplantation and then returned to normal growth.

FIG. 2B illustrates that pre-intervention microbiota recipients showed significantly greater fat mass as a percentage of body weight.

FIG. 2C illustrates that adipocytes from mice receiving the post-intervention microbiota did not change over time.

FIG. 2D-F illustrate RT-qPCR of TNFα, IL6 and TLR4 gene expression in liver, ileum and colon.

FIG. 3A and FIG. 3B illustrate that the composition of the gut microbiota showed a significant shift after 30 days of the intervention in both cohorts as indicated by principal coordinates analysis (PCoA, multivariate analysis of variance, (MANOVA) test, P=2.17e-6) based on Bray-Curtis dissimilarity of the 376 bacterial CAGs.

FIG. 3C illustrates that ward clustering algorithm and Permutational MANOVA (9999 permutations, P<0.001) based on bootstrapped Spearman correlation coefficients clustered these bacterial CAGs into 18 co-abundance species/strains (CAS) groups.

FIG. 3D illustrates that the agreement between strain-level and CAS-level procrustes analysis with host bioclinical variables.

FIG. 3E illustrates that 6 CASs, including CAS13 containing the most predominant species Prevotellacopri, did not change their abundance after the intervention (data not shown). CAS1, 3 and 4 significantly increased their abundance after the intervention while CAST, 8, 11, 12, 14, 15, 16, 17 and 18 decreased.

FIG. 4A and FIG. 4B illustrate that the PCA score plot of all the KOs showed a significant shift after the intervention.

FIG. 4C illustrates that metabolic profiling of fecal water indicates a shift from fat and protein fermentation to carbohydrate fermentation in the gut after the intervention, in agreement with the identified changes of KEGG pathways.

FIG. 5 illustrates that gene richness in the gut microbiota is decreased after the intervention. The change of gene counts adjusted to 28 million mapped reads per sample in PWS and SO subjects. Data are mean±s.e.m. Wilcoxon matched-pairs signed rank test (two-tailed) for each pair-wise comparison in PWS or SO children. * P<0.05, ** P<0.01, *** P<0.001.

FIG. 6 illustrates that structural changes in the gut microbiota are significantly associated with improved biomedical parameters. Procrustes analysis combining PCoA (base on Bray-Curtis distance) of 376 bacterial CAGs (end of lines with solid symbols) with PCA of bioclinical variables presented in FIG. 1 (end of lines without solid symbols). For PWS, n=17 at Day 0, 30, 60, and 90; For SO, n=21 at Day 0 and n=20 at Day 30.

FIG. 7 illustrates that total fecal bacteria is reduced after the dietary intervention. qPCR was used to measure the copy number of the V3 region in 16S rRNA gene from fecal bacteria. Data are mean±s.e.m. Wilcoxon matched-pairs signed rank test (two-tails) was used to analyze variation between each two time points in PWS or SO children. Mann-Whitney U test (two-tails) was used to analyze variation between PWS and SO children at baseline or 30 days after intervention. * P<0.05, ** P<0.01. For PWS, n=17; For SO, n=21.

FIG. 8 illustrates that compared to pro-intervention, Band HAL Band HA7 and Band HA12 were significantly enriched along with intervention and became main bands at 105th day.

### DETAILED DESCRIPTION OF THE INVENTION

The present inventors have discovered strains of B. pseudocatenulatum that can reduce simple or genetic obesity, alleviate metabolic deteriorations, and reduce inflammation and fat accumulation in mammals. The B. pseudocatenulatum strains of the present invention, alone or in combination with other probiotic microorganisms, when established in the gut, function as foundation species that define the structure of a healthy gut ecosystem, for example by rendering the gut environment unfavorable to pathogenic and detrimental bacteria, possibly via increased production of acetate.

As described in more details below, the B. pseudocatenulatum strains of the present invention were isolated from individuals subjected to hospitalized intervention with a previous published diet based on whole-grains, traditional Chinese medicinal foods and prebiotics (WTP diet) (S. Xiao et al., A gut microbiota-targeted dietary intervention for amelioration of chronic inflammation underlying metabolic syndrome. FEMS Microbiol Ecol 87, 357 (February, 2014). These individuals, after the intervention, have shown a significant alleviation of the metabolic deteriorations in children with both genetic and simple obesity after 30 days of the dietary intervention.

As detailed in the Examples below, the present inventors, using a combination of traditional bacterial isolation methodologies, denatured gradient gel electrophoresis (DGGE), ERIC-PCR, 16S rRNA sequencing, and whole genome technologies, and successfully obtained a large number of strains of the foundation species of the present invention, identified as B. pseudocatenulatum. A representative isolate is the C95 strain, deposited in the China General Microbiological Culture Collection Center (CGMCC) on Feb. 9, 2015, with the accession no. of CGMCC10549.

In one embodiment, the probiotic strain of the present invention comprises a genome which, in comparison to that of the C95, has a percent query coverage of at least 81%, preferably at least 88%, more preferably at least 88.5% percent. Further, the aligned regions share at least 98.5% sequence identity, preferably at least 99% sequence identity.

The probiotic strains of the present invention can be cultured, maintained and propagated using established methods well-known to those ordinarily skilled in the art, some of which methods are exemplified in the Examples below.

The bacterium used in the present invention is a Bifidobacterium pseudocatenulatum strain or a mixture thereof. Preferably the Bifidobacterium to be used in the present invention is a B. pseudocatenulatum C95 strain.

The bacterium may be used in any form capable of exerting the effects described herein. Preferably, the bacteria are viable bacteria.

The bacteria may comprise whole bacteria or may comprise bacterial components. Examples of such components include bacterial cell wall components such as peptidoglycan, bacterial nucleic acids such as DNA and RNA, bacterial membrane components, and bacterial structural components such as proteins, carbohydrates, lipids and combinations of these such as lipoproteins, glycolipids and glycoproteins.

The bacteria may also or alternatively comprise bacterial metabolites. In this specification the term ‘bacterial metabolites’ includes all molecules produced or modified by the (probiotic) bacteria as a result of bacterial metabolism during growth, survival, persistence, transit or existence of bacteria during probiotic product manufacture and storage and during gastrointestinal transit in a mammal. Examples include all organic acids, inorganic acids, bases, proteins and peptides, enzymes and co-enzymes, amino acids and nucleic acids, carbohydrates, lipids, glycoproteins, lipoproteins, glycolipids, vitamins, all bioactive compounds, metabolites containing an inorganic component, and all small molecules, for example nitrous molecules or molecules containing a sulphurous acid. Preferably the bacteria comprise whole bacteria, more preferably whole viable bacteria.

Preferably, the Bifidobacterium used in accordance with the present invention is one which is suitable for human and/or animal consumption. In the present invention, the Bifidobacterium used may be of the same type (species and strain) or may comprise a mixture of species and/or strains.

Suitable Bifidobacteria are selected from the species Bifidobacterium lactis, Bifidobacterium bifidium, Bifidobacterium longum, Bifidobacterium animalis, Bifidobacterium breve, Bifidobacterium infantis, Bifidobacterium catenulatum, Bifidobacterium pseudocatenulatum, Bifidobacterium adolescentis, and Bifidobacterium angulatum, and combinations of any thereof.

As shown in the Examples below, Lactobacillus mucosae, especially those that are highly similar to L. mucosae strain 32, were highly increased post diet intervention. Thus, one preferred bacterium for use in combination with a B. pseudocatenulatum strain of the present invention is L. mucosae, especially Strain 32.

In one embodiment, the bacterium used in the present invention is a probiotic bacterium. In this specification the term ‘probiotic bacterium’ is defined as covering any non-pathogenic bacterium which, when administered live in adequate amounts, confer a health benefit on the host. These probiotic strains generally have the ability to survive the passage through the upper part of the digestive tract. They are non-pathogenic, non-toxic and exercise their beneficial effect on health on the one hand via ecological interactions with the resident flora in the digestive tract, and on the other hand via their ability to influence the immune system in a positive manner via the “GALT” (gut-associated lymphoid tissue). Depending on the definition of probiotics, these bacteria, when given in a sufficient number, have the ability to progress live through the intestine, however they do not cross the intestinal barrier and their primary effects are therefore induced in the lumen and/or the wall of the gastrointestinal tract. They then form part of the resident flora during the administration period. This colonization (or transient colonization) allows the probiotic bacteria to exercise a beneficial effect, such as the repression of potentially pathogenic micro-organisms present in the flora and interactions with the immune system of the intestine.

In some embodiments, the Bifidobacterium is used in the present invention together with a bacterium of the genus Lactobacillus. A combination of Bifidobacterium and Lactobacillus bacteria according to the present invention exhibits a synergistic effect in certain applications (i.e. an effect which is greater than the additive effect of the bacteria when used separately). For example, combinations which, in addition to having effect on the mammal as single components, may have beneficial effect on the other components of the combination, for example by producing metabolites which are then in turn used as an energy source by other components of the combination, or maintaining physiological conditions which favour the other components.

Typically, the Lactobacillus bacteria are selected from the species Lactobacillus acidophilus, Lactobacillus casei, Lactobacillus kefiri, Lactobacillus bifidus, Lactobacillus brevis, Lactobacillus helveticus, Lactobacillus paracasei, Lactobacillus rhamnosus, Lactobacillus salivarius, Lactobacillus curvatus, Lactobacillus bulgaricus, Lactobacillus sakei, Lactobacillus reuteri, Lactobacillus fermentum, Lactobacillus farciminis, Lactobacillus lactis, Lactobacillus delbreuckii, Lactobacillus plantarum, Lactobacillus paraplantarum, Lactobacillus crispatus, Lactobacillus gassed, Lactobacillus johnsonii and Lactobacillus jensenii, and combinations of any thereof.

In preferred embodiments, the Lactobacillus bacterium used in the present invention is a probiotic Lactobacillus. Preferably, the Lactobacillus bacterium used in the present invention of the species Lactobacillus acidophilus.

Dosage and Administration.

Administration of probiotic bacteria can be accomplished by any method likely to introduce the organisms into the digestive tract. The bacteria can be mixed with a carrier and applied to liquid or solid feed or to drinking water. The carrier material should be non-toxic to the bacteria and the animal. Preferably, the carrier contains an ingredient that promotes viability of the bacteria during storage. The bacteria can also be formulated as an inoculant paste to be directly injected into an animal's mouth. The formulation can include added ingredients to improve palatability, improve shelf-life, impart nutritional benefits, and the like. If a reproducible and measured dose is desired, the bacteria can be administered by a rumen cannula. The amount of probiotic bacteria to be administered is governed by factors affecting efficacy. When administered in feed or drinking water the dosage can be spread over a period of days or even weeks. The cumulative effect of lower doses administered over several days can be greater than a single larger dose thereof. By monitoring the numbers of Salmonella strains that cause human salmonellosis in feces before, during and after administration of dominant probiotic bacteria, those skilled in the art can readily ascertain the dosage level needed to reduce the amount of Salmonella strains that cause human salmonellosis carried by the animals. One or more strains of dominant probiotic bacteria can be administered together. A combination of strains can be advantageous because individual animals may differ as to the strain which is most persistent in a given individual.

The Bifidobacterium pseudocatenulatum used in accordance with the present invention may comprise from 10⁶ to 10¹² CFU of bacteria/g of support, and more particularly from 10⁸ to 10¹² CFU of bacteria/g of support, preferably 10⁹ to 10¹² CFU/g for the lyophilized form.

Suitably, the B. pseudocatenulatum may be administered at a dosage of from about 10⁶ to about 10¹² CFU of microorganism/dose, preferably about 10⁸ to about 10¹² CFU of microorganism/dose. By the term “per dose” it is meant that this amount of microorganism is provided to a subject either per day or per intake, preferably per day. For example, if the microorganism is to be administered in a food product (for example, in yoghurt)—then the yoghurt will preferably contain from about 10⁸ to 10¹² CFU of the microorganism. Alternatively, however, this amount of microorganism may be split into multiple administrations each consisting of a smaller amount of microbial loading—so long as the overall amount of microorganism received by the subject in any specific time (for instance each 24 hour period) is from about 10⁶ to about 10¹² CFU of microorganism, preferably 10⁸ to about 10¹² CFU of microorganism.

In accordance with the present invention an effective amount of at least one strain of a microorganism may be at least 10⁶ CFU of microorganism/dose, preferably from about 10⁶ to about 10¹² CFU of microorganism/dose, preferably about 10⁸ to about 10¹² CFU of microorganism/dose.

In one embodiment, the B. pseudocatenulatum strain may be administered at a dosage of from about 10⁶ to about 10¹² CFU of microorganism/day, preferably about 10⁸ to about 10¹² CFU of microorganism/day. Hence, the effective amount in this embodiment may be from about 10⁶ to about 10¹² CFU of microorganism/day, preferably about 10⁸ to about 10¹² CFU of microorganism/day.

CFU stands for “colony-forming units”. By “support” is meant the food product, dietary supplement or the pharmaceutically acceptable support.

When Bifidobacteria are used in the present invention together with another probiotic bacterium, the bacteria may be present in any ratio capable of achieving the desired effects of the invention described herein.

### Subjects/Medical Indications

The B. pseudocatenulatum strain is administered to a mammal, including for example livestock (including cattle, horses, pigs, chickens and sheep), and humans. In some aspects of the present invention the mammal is a companion animal (including pets), such as a dog or a cat for instance. In some aspects of the present invention, the subject may suitably be a human.

The B. pseudocatenulatum strain may be suitable for treating a number of diseases or conditions in mammals (particularly humans). In this specification the term “treatment” or “treating” refers to any administration of the B. pseudocatenulatum strain of the present invention in (1) preventing the specified disease from occurring in a mammal which may be predisposed to the disease but does not yet experience or display the pathology or symptomatology of the disease (including prevention of one or more risk factors associated with the disease); (2) inhibiting the disease in a mammal that is experiencing or displaying the pathology or symptomatology of the diseased, or (3) ameliorating the disease in a mammal that is experiencing or displaying the pathology or symptomatology of the diseased.

The B. pseudocatenulatum strain of the present invention is suitable for administration to both diabetic and obese mammals. They could also be suitable for diabetic and non-obese mammals, as well as to obese mammals possessing the risk factors for diabetes, but not yet in a diabetic state. This aspect is discussed in more detail below.

As described in more detail in the Examples below, the B. pseudocatenulatum strain of the present invention has a number of biological activities. In particular, the Bifidobacteria used in the present invention are capable of normalising insulin sensitivity, increasing fed insulin secretion, decreasing fasted insulin secretion, improving glucose tolerance in a mammal. These effects confer the potential for use in the treatment of diabetes and diabetes-related conditions (in particular, Type 2 diabetes and impaired glucose tolerance).

In addition, the Bifidobacteria used in the present invention are capable of inducing weight loss and lowering body fat mass (in particular, mesenteric fat mass). These effects confer the potential for use in the treatment of obesity and controlling weight gain and/or inducing weight loss in a mammal.

In particular, as described in more detail in the Examples below, the Bifidobacteria used in combination with Lactobacillus bacteria (particularly Lactobacillus acidophilus bacteria) in accordance with the present invention are capable of inducing weight loss and lowering body fat mass (in particular, mesenteric fat mass). These effects confer the potential for use in the treatment of obesity and controlling weight gain and/or inducing weight loss in a mammal.

In this specification, the term obesity is linked to body mass index (BMI). The body mass index (BMI) (calculated as weight in kilograms divided by the square of height in metres) is the most commonly accepted measurement for overweight and/or obesity. A BMI exceeding 25 is considered overweight. Obesity is defined as a BMI of 30 or more, with a BMI of 35 or more considered as serious comorbidity obesity and a BMI of 40 or more considered morbid obesity.

As noted above, the term “obesity” as used herein includes obesity, comorbidity obesity and morbid obesity. Therefore, the term “obese” as used here may be defined as a subject having a BMI of more than or equal to 30. In some embodiments, suitably an obese subject may have a BMI of more than or equal to 30, suitably 35, suitably 40.

While the composition of the invention is particularly suitable for use in patients who are both diabetic and obese, the composition is also suitable for those who are diabetic but not obese. It may also be suitable for use in obese patients possessing the risk factors for diabetes, but not yet in a diabetic state, as it could be expected that an obese person (but not diabetic), could limit the metabolic consequences of his obesity, i.e. the diabetes or at least insulino-resistance development.

In addition, the Bifidobacteria used in the present invention may be used for treating metabolic syndrome in a mammal. Metabolic syndrome is a combination of medical disorders that increase the risk of developing cardiovascular disease and diabetes. Metabolic syndrome is also known as metabolic syndrome X, syndrome X, insulin resistance syndrome, Reaven's syndrome or CHAOS (Australia).

### Genetic Obesity

In further embodiments, the Bifidobacteria (and, if present, the Lactobacilli) used in the present invention may be used to lower tissue inflammation (particularly, although not exclusively, liver tissue inflammation, muscle tissue inflammation and/or adipose tissue inflammation) in a mammal.

Examples of cardiovascular diseases treatable by use of the Bifidobacteria (and, if present, the Lactobacilli) according to the present invention include aneurysm, angina, atherosclerosis, cerebrovascular accident (stroke), cerebrovascular disease, congestive heart failure (CHF), coronary artery disease, myocardial infarction (heart attack) and peripheral vascular disease.

It is envisaged within the scope of the present invention that the embodiments of the invention can be combined such that combinations of any of the features described herein are included within the scope of the present invention. In particular, it is envisaged within the scope of the present invention that any of the therapeutic effects of the bacteria may be exhibited concomitantly.

### Compositions

While is it possible to administer the B. pseudocatenulatum strain of the present invention alone according to the present invention (i.e. without any support, diluent or excipient), the B. pseudocatenulatum strain of the present invention is typically and preferably administered on or in a support as part of a product, in particular as a component of a food product, a dietary supplement or a pharmaceutical formulation. These products typically contain additional components well known to those skilled in the art.

Any product which can benefit from the composition may be used in the present invention. These include but are not limited to foods, particularly fruit conserves and dairy foods and dairy food-derived products, and pharmaceutical products. The B. pseudocatenulatum strain of the present invention may be referred to herein as “the composition of the present invention” or “the composition”.

### Food

In one embodiment, the B. pseudocatenulatum strain of the present invention is employed in a food product such as a food supplement, a drink or a powder based on milk. Here, the term “food” is used in a broad sense and covers food for humans as well food for animals (i.e. a feed). In a preferred aspect, the food is for human consumption.

The food may be in the form of a solution or as a solid—depending on the use and/or the mode of application and/or the mode of administration. When used as, or in the preparation of a food, such as functional food, the composition of the present invention may be used in conjunction with one or more of: a nutritionally acceptable carrier, a nutritionally acceptable diluent, a nutritionally acceptable excipient, a nutritionally acceptable adjuvant, a nutritionally active ingredient.

By way of example, the composition of the present invention can be used as an ingredient to soft drinks, a fruit juice or a beverage comprising whey protein, health teas, cocoa drinks, milk drinks and lactic acid bacteria drinks, yoghurt and drinking yoghurt, cheese, ice cream, water ices and desserts, confectionery, biscuits cakes and cake mixes, snack foods, balanced foods and drinks, fruit fillings, care glaze, chocolate bakery filling, cheese cake flavoured filling, fruit flavoured cake filling, cake and doughnut icing, instant bakery filling creams, fillings for cookies, ready-to-use bakery filling, reduced calorie filling, adult nutritional beverage, acidified soy/juice beverage, aseptic/retorted chocolate drink, bar mixes, beverage powders, calcium fortified soy/plain and chocolate milk, calcium fortified coffee beverage.

The composition can further be used as an ingredient in food products such as American cheese sauce, anti-caking agent for grated & shredded cheese, chip dip, cream cheese, dry blended whip topping fat free sour cream, freeze/thaw dairy whipping cream, freeze/thaw stable whipped topping, low fat and light natural cheddar cheese, low fat Swiss style yoghurt, aerated frozen desserts, hard pack ice cream, label friendly, improved economics & indulgence of hard pack ice cream, low fat ice cream: soft serve, barbecue sauce, cheese dip sauce, cottage cheese dressing, dry mix Alfredo sauce, mix cheese sauce, dry mix tomato sauce and others.

The term “dairy product” as used herein is meant to include a medium comprising milk of animal and/or vegetable origin. As milk of animal origin there can be mentioned cow's, sheep's, goat's or buffalo's milk. As milk of vegetable origin there can be mentioned any fermentable substance of vegetable origin which can be used according to the invention, in particular originating from soybeans, rice or cereals.

For certain aspects, preferably the present invention may be used in connection with yoghurt production, such as fermented yoghurt drink, yoghurt, drinking yoghurt, cheese, fermented cream, milk based desserts and others.

Suitably, the composition can be further used as an ingredient in one or more of cheese applications, meat applications, or applications comprising protective cultures.

The present invention also provides a method of preparing a food or a food ingredient, the method comprising admixing the composition according to the present invention with another food ingredient.

Advantageously, the present invention relates to products that have been contacted with the composition of the present invention (and optionally with other components/ingredients), wherein the composition is used in an amount to be capable of improving the nutrition and/or health benefits of the product.

As used herein the term “contacted” refers to the indirect or direct application of the composition of the present invention to the product. Examples of the application methods which may be used, include, but are not limited to, treating the product in a material comprising the composition, direct application by mixing the composition with the product, spraying the composition onto the product surface or dipping the product into a preparation of the composition.

Where the product of the invention is a foodstuff, the composition of the present invention is preferably admixed with the product. Alternatively, the composition may be included in the emulsion or raw ingredients of a foodstuff. In a further alternative, the composition may be applied as a seasoning, glaze, colorant mixture, and the like.

The compositions of the present invention may be applied to intersperse, coat and/or impregnate a product with a controlled amount of a microorganism.

Preferably, the composition is used to ferment milk or sucrose fortified milk or lactic media with sucrose and/or maltose where the resulting media containing all components of the composition—i.e. said microorganism according to the present invention—can be added as an ingredient to yoghurt milk in suitable concentrations such as for example in concentrations in the final product which offer a daily dose of 10⁶-10¹⁰ cfu. The microorganism according to the present invention may be used before or after fermentation of the yoghurt.

For some aspects the microorganisms according to the present invention are used as, or in the preparation of, animal feeds, such as livestock feeds, in particular poultry (such as chicken) feed, or pet food.

Advantageously, where the product is a food product, the B. pseudocatenulatum strain of the present invention should remain effective through the normal “sell-by” or “expiration” date during which the food product is offered for sale by the retailer. Preferably, the effective time should extend past such dates until the end of the normal freshness period when food spoilage becomes apparent. The desired lengths of time and normal shelf life will vary from foodstuff to foodstuff and those of ordinary skill in the art will recognize that shelf-life times will vary upon the type of foodstuff, the size of the foodstuff, storage temperatures, processing conditions, packaging material and packaging equipment.

### Food Ingredient, Food Supplements, and Functional Foods

The composition of the present invention may be used as a food ingredient and/or feed ingredient. As used herein the term “food ingredient” or “feed ingredient” includes a formulation which is or can be added to functional foods or foodstuffs as a nutritional supplement. The food ingredient may be in the form of a solution or as a solid—depending on the use and/or the mode of application and/or the mode of administration

The composition of the present invention may be—or may be added to—food supplements (also referred to herein as dietary supplements).

The composition of the present invention may be—or may be added to—functional foods. As used herein, the term “functional food” means food which is capable of providing not only a nutritional effect, but is also capable of delivering a further beneficial effect to consumer.

Accordingly, functional foods are ordinary foods that have components or ingredients (such as those described herein) incorporated into them that impart to the food a specific functional—e.g. medical or physiological benefit—other than a purely nutritional effect. Some functional foods are nutraceuticals. Here, the term “nutraceutical” means a food which is capable of providing not only a nutritional effect and/or a taste satisfaction, but is also capable of delivering a therapeutic (or other beneficial) effect to the consumer. Nutraceuticals cross the traditional dividing lines between foods and medicine.

### Medicament

The term “medicament” as used herein encompasses medicaments for both human and animal usage in human and veterinary medicine. In addition, the term “medicament” as used herein means any substance which provides a therapeutic and/or beneficial effect. The term “medicament” as used herein is not necessarily limited to substances which need Marketing Approval, but may include substances which can be used in cosmetics, nutraceuticals, food (including feeds and beverages for example), probiotic cultures, and natural remedies. In addition, the term “medicament” as used herein encompasses a product designed for incorporation in animal feed, for example livestock feed and/or pet food.

### Pharmaceuticals

The composition of the present invention may be used as—or in the preparation of—a pharmaceutical. Here, the term “pharmaceutical” is used in a broad sense—and covers pharmaceuticals for humans as well as pharmaceuticals for animals (i.e. veterinary applications). In a preferred aspect, the pharmaceutical is for human use and/or for animal husbandry. The pharmaceutical can be for therapeutic purposes—which may be curative or palliative or preventative in nature. The pharmaceutical may even be for diagnostic purposes.

A pharmaceutically acceptable support may be for example a support in the form of compressed tablets, tablets, capsules, ointments, suppositories or drinkable solutions. Other suitable forms are provided below.

When used as—or in the preparation of—a pharmaceutical, the composition of the present invention may be used in conjunction with one or more of: a pharmaceutically acceptable carrier, a pharmaceutically acceptable diluent, a pharmaceutically acceptable excipient, a pharmaceutically acceptable adjuvant, a pharmaceutically active ingredient. The pharmaceutical may be in the form of a solution or as a solid—depending on the use and/or the mode of application and/or the mode of administration.

Examples of nutritionally acceptable carriers for use in preparing the forms include, for example, water, salt solutions, alcohol, silicone, waxes, petroleum jelly, vegetable oils, polyethylene glycols, propylene glycol, liposomes, sugars, gelatin, lactose, amylose, magnesium stearate, talc, surfactants, silicic acid, viscous paraffin, perfume oil, fatty acid monoglycerides and diglycerides, petroethral fatty acid esters, hydroxymethylcellulose, polyvinylpyrrolidone, and the like.

For aqueous suspensions and/or elixirs, the composition of the present invention may be combined with various sweetening or flavouring agents, colouring matter or dyes, with emulsifying and/or suspending agents and with diluents such as water, propylene glycol and glycerin, and combinations thereof. The forms may also include gelatin capsules; fibre capsules, fibre tablets etc.; or even fibre beverages. Further examples of form include creams. For some aspects the microorganism used in the present invention may be used in pharmaceutical and/or cosmetic creams such as sun creams and/or after-sun creams for example.

Combinations with Prebiotics

The composition of the present invention may additionally contain one or more prebiotics. Prebiotics are a category of functional food, defined as non-digestible food ingredients that beneficially affect the host by selectively stimulating the growth and/or activity of one or a limited number of bacteria in the colon, and thus improve host health. Typically, prebiotics are carbohydrates (such as oligosaccharides), but the definition does not preclude non-carbohydrates. The most prevalent forms of prebiotics are nutritionally classed as soluble fibre. To some extent, many forms of dietary fibre exhibit some level of prebiotic effect.

In one embodiment, a prebiotic is a selectively fermented ingredient that allows specific changes, both in the composition and/or activity in the gastrointestinal microflora that confers benefits upon host well-being and health.

Suitably, the prebiotic may be used according to the present invention in an amount of 0.01 to 100 g/day, preferably 0.1 to 50 g/day, more preferably 0.5 to 20 g/day. In one embodiment, the prebiotic may be used according to the present invention in an amount of 1 to 100 g/day, preferably 2 to 9 g/day, more preferably 3 to 8 g/day. In another embodiment, the prebiotic may be used according to the present invention in an amount of 5 to 50 g/day, preferably 10 to 25 g/day.

Examples of dietary sources of prebiotics include soybeans, inulin sources (such as Jerusalem artichoke, jicama, and chicory root), raw oats, unrefined wheat, unrefined barley and yacon. Examples of suitable prebiotics include alginate, xanthan, pectin, locust bean gum (LBG), inulin, guar gum, galacto-oligosaccharide (GOS), fructo-oligosaccharide (FOS), polydextrose (i.e. Litesse®), lactitol, lactosucrose, soybean oligosaccharides, isomaltulose (Palatinose™), isomalto-oligosaccharides, gluco-oligosaccharides, xylo-oligosaccharides, manno-oligosaccharides, beta-glucans, cellobiose, raffinose, gentiobiose, melibiose, xylobiose, cyclodextrins, isomaltose, trehalose, stachyose, panose, pullulan, verbascose, galactomannans, and all forms of resistant starches. A particularly preferred example of a prebiotic is polydextrose.

In some embodiments, a combination of the B. pseudocatenulatum strain of the present invention and prebiotics according to the present invention exhibits a synergistic effect in certain applications (i.e. an effect which is greater than the additive effect of the bacteria when used separately).

### EXAMPLES

### Example 1 Dietary Alleviation of Genetic and Simple Obesity

1. Dietary Intervention Alleviated Genetic and Simple Obesity, and Improved Bio-Clinical Parameters of Simple or Genetic Obesity Patients

The WTP diet (14) was used for this hospitalized intervention study performed on morbidly obese children with PWS or SO. The two cohorts (SO, n=21, average age 10.52 yrs, range 3-16 yrs; PWS, n=17, average age 9.26 yrs, range 5-16 yrs) showed no significant difference in age range (data not shown). Both cohorts received the hospitalized intervention for 30 days. Due to the requirements of the parents, the PWS cohort continued for another 60 days. One volunteer (GD02) stayed in the hospital for 285 days. During the dietary intervention, both cohorts of children reduced their total calorie intake by about 30% compared to their pre-intervention diets. Protein intake remained at 13-14% of total kcal consumed. Carbohydrate intake increased from 52% to 62% of total calories in PWS and from 57% to 62% in SO. The form of carbohydrates changed from primarily white rice and wheat flour to whole grains. Fat intake decreased from 34% to 20% of total calories in PWS and from 30% to 20% in SO. The most substantial change was the total dietary fiber intake, which increased from 6 g to 49 g per day in PWS and from 9 g to 51 g per day in SO (data not shown). Anthropometric measurements and metabolic panel blood testing were used to track changes.

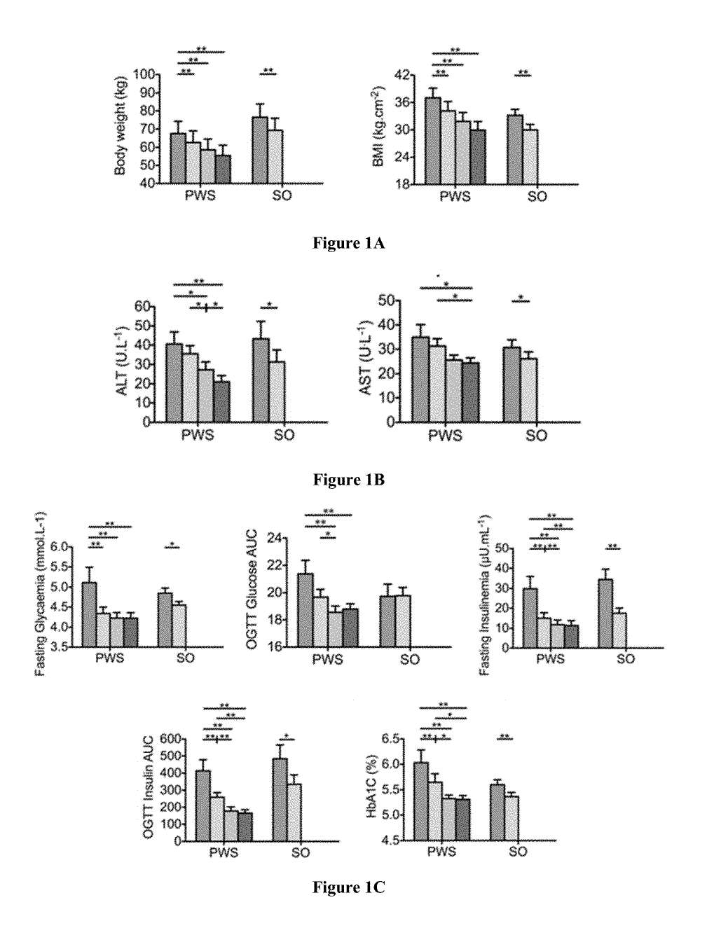

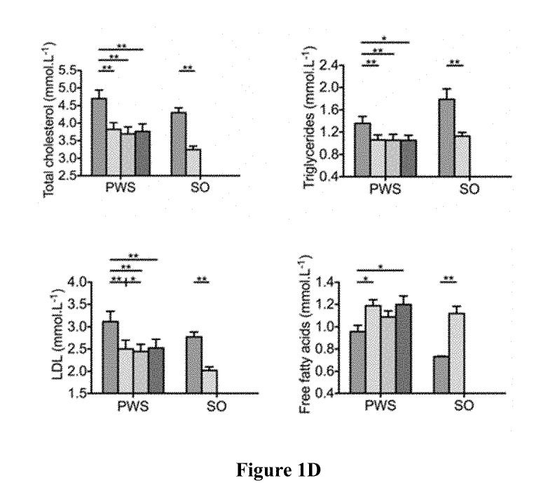

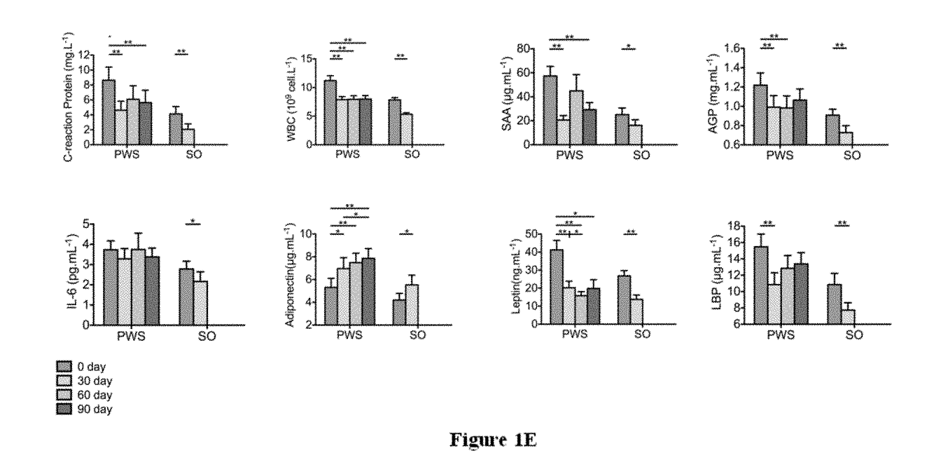

All relevant bioclinical parameters indicated a significant alleviation of the metabolic deteriorations in children with both genetic and simple obesity after 30 days of the dietary intervention (FIG. 1). After 30 days of intervention, the SO cohort lost 9.5±0.4% (mean±s.e.m.) of their initial bodyweight, and the PWS cohort lost 7.6±0.6% (FIG. 1A). Both PWS and SO children showed significant improvement in markers of metabolic health (data not shown). Aspartate aminotransferase (AST) and alanine aminotransferase (ALT) levels in the blood were reduced, indicating improved liver condition (FIG. 1B). Glucose homeostasis was improved, indicating better insulin sensitivity (FIG. 1C). Blood levels of total cholesterol, triglycerides, and low-density lipoprotein (LDL) were decreased (FIG. 1D). The PWS cohort was followed for two more months on the WTP diet. They lost a total of 18.3±1.0% of their initial bodyweight and showed continued improvement in several metabolic parameters (FIG. 1A-D). In addition, the PWS cohort showed a modest improvement in their overall hyperphagia behavior (data not shown). GD02 reduced his bodyweight from 140.1 kg to 83.6 kg after 285 days in the hospital. He then continued this intervention at home and reduced to 73 kg after 430 days on this diet. All his metabolic parameters came to normal range (data not shown). This extended dietary intervention can thus significantly alleviate the metabolic deteriorations in human genetic obesity, in which the diet-induced weight loss can be comparable to that achievable by gastric bypass surgery (18).

Several markers of systemic inflammation were also improved in PWS and SO cohorts after 30 days of dietary intervention, including C-reactive protein (CRP), serum amyloid A protein (SAA), α-acid glycoprotein (AGP) and white blood cell count (WBC) (FIG. 1E). The level of adiponectin, an anti-inflammatory adipokine, was increased, and leptin was decreased, indicating an alleviation of “at-risk” phenotype (19). Lipopolysaccharide binding protein (LBP), a surrogate marker for bacterial endotoxin in the blood (20), was decreased (FIG. 1E). Since endotoxin and its bacterial producers have been mechanistically linked with development of obesity and insulin resistance (15, 21), the reduced endotoxin load and inflammation in PWS and SO children suggests that both cohorts have a healthier gut microbiota with lower production of proinflammatory antigens such as endotoxins after the intervention.

2. Post Intervention Gut Microbiota Induced Less Inflammation and Fat Deposition in Mice

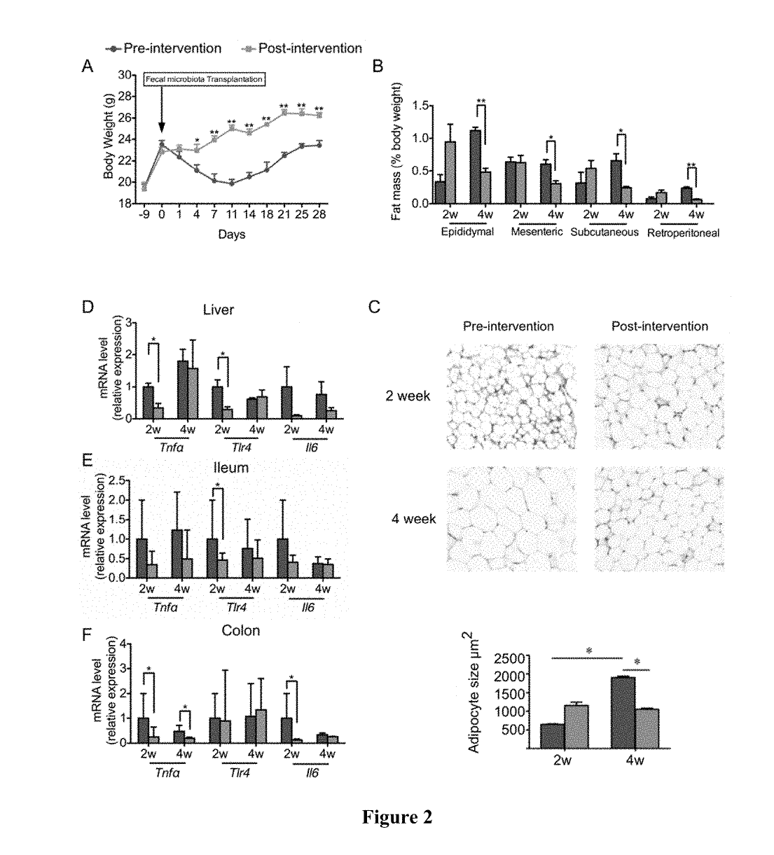

To compare the capacity of gut microbiota to induce metabolic deteriorations before and after the intervention, we transplanted the gut microbiota from the same PWS volunteer (GD58) before (Day 0) and after (Day 90) the intervention, into germ-free wild-type C57BL/6J mice. Mice that received the pre-intervention human fecal microbiota showed significantly decreased bodyweight during the first two weeks after transplantation, suggesting toxicity from the transplant, and then regained the lost weight in the following two weeks. Mice that received the post-intervention human fecal microbiota lost no bodyweight. Rather, they maintained weight for 4 days after transplantation and then returned to normal growth (FIG. 2A). Interestingly, although the total bodyweight of the mice receiving the pre-intervention microbiota was still significantly lower than that of mice receiving the post-intervention transplant by the end of the trial, pre-intervention microbiota recipients showed significantly greater fat mass as a percentage of body weight (FIG. 2B). Histological examination of epididymal fat pads revealed that the mean cell area of adipocytes in mice that received the pre-intervention gut microbiota was smaller than in post-intervention recipients at 2 weeks after the transplantation, consistent with toxicity of the microbiota, but then increased significantly at the end of the trial. Adipocytes from mice receiving the post-intervention microbiota did not change over time (FIG. 2C). The initial weight loss was associated with appreciably higher inflammatory responses in pre-intervention transplant recipients, as measured by RT-qPCR of TNFα, IL6 and TLR4 gene expression in liver, ileum and colon at 2 weeks after transplantation (FIG. 2D-F). These data suggest that the pre-intervention gut microbiota from the PWS patient indeed had a greater capacity to induce inflammation and fat deposition in mice than the post-intervention.

### Dietary Intervention Allowed Establishment of Beneficial Foundation Species Bifidobacterium Pseudocatenulatum in the Gut Microbiota

Several structural patterns of the gut microbial community have been associated with obesity, such as a high Firmicutes/Bacteroidetes ratio and low gene richness, but the specific relevant members of the gut microbiota and their functional interactions that contribute to obesity development and associated metabolic deteriorations require further characterization (17, 22-25).

To determine how the overall structure of the gut microbiota was modulated during the dietary intervention, we performed shotgun metagenomic sequencing on fecal samples from both cohorts and analyzed the data using the recently developed “canopy-based” algorithm, which segregates individual genes into co-abundance gene (CAG) groups based on the fact that the abundances of two genes encoded by the same genomic DNA molecule will highly correlate with each other across complex metagenomic samples (26). With sufficient sequencing depth, reads in a CAG can be assembled into a draft genome, which allowed us to perform genome-specific, strain-level analysis of microbiota changes induced by the dietary intervention.

Using the Illumina Hiseq 2000 platform, we performed shotgun metagenomic sequencing on 110 fecal samples collected from 21 SO (Day 0 and 30) and 17 PWS (Day 0, 30, 60, and 90) subjects. On average, 76.0±18.0 million (mean±s.d.) high-quality paired-end reads from each sample were used for de novo assembly and gene prediction (data not shown). The non-redundant gene catalogue of 2,077,766 microbial genes was constructed. These two million genes were binned into 28,072 CAGs using the canopy-based algorithm with a high cutoff for correlation coefficients (>0.9) to maximize chances that genes of a CAG were from the same genome (26). 376CAGseach with >700 genes were considered as bacterial genomes of individual strains, which accounted for 36.4% (775,515) of the recognized genes. Of the 376 CAGs, we focused our subsequent analyses on 161 that were shared by at least 20% of the samples. The 161 prevalent CAGs were assembled into draft genomes, and 118 of the genome assemblies met at least five of the six quality criteria of the Human Microbiome Project for standard reference genomes (data not shown). Fifty of the assemblies were closely related to known reference genomes with coverage over 80% and identity over 95% (data not shown). Ten species had more than one draft genome assembled, e.g. Faecalibacterium prausnitzii having nine assembled genomes, and Eubacterium eligens having five, showing strain-level diversity in these species.

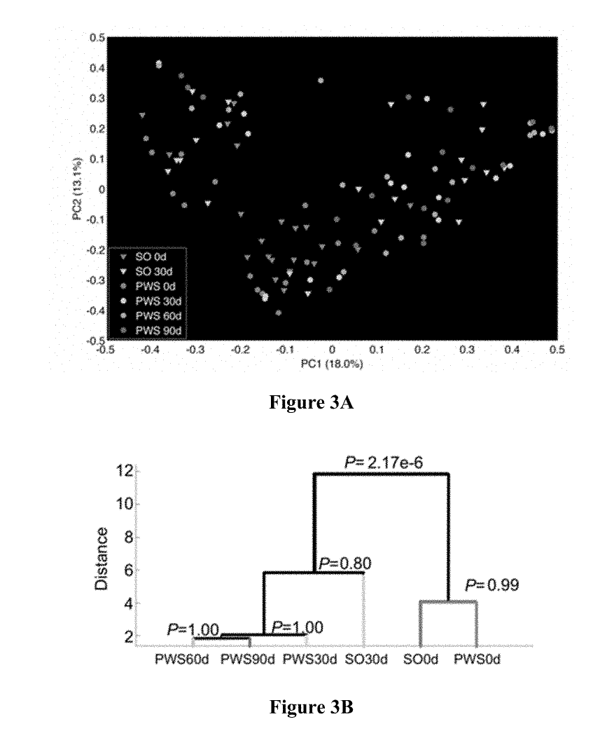

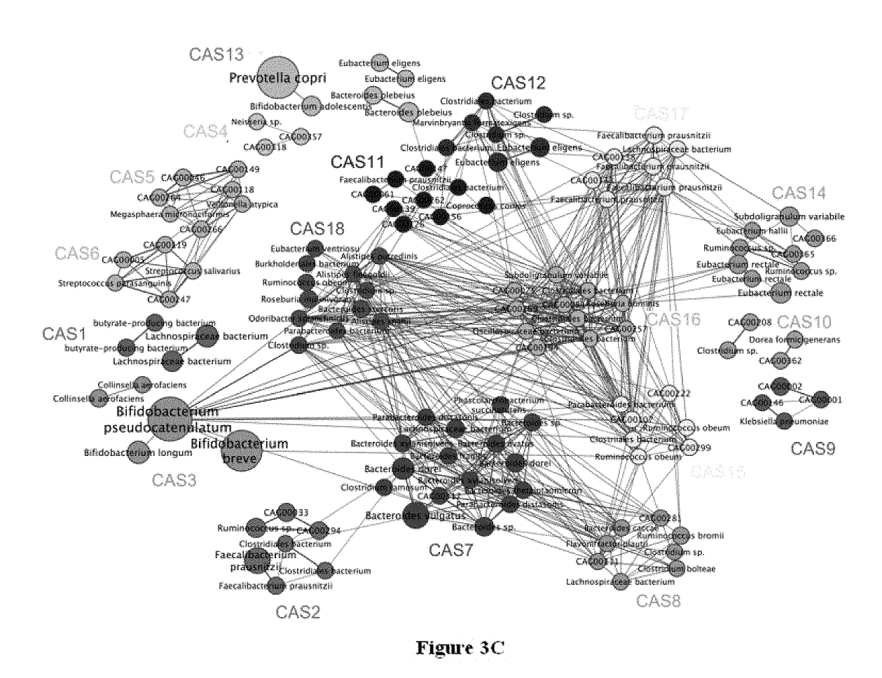

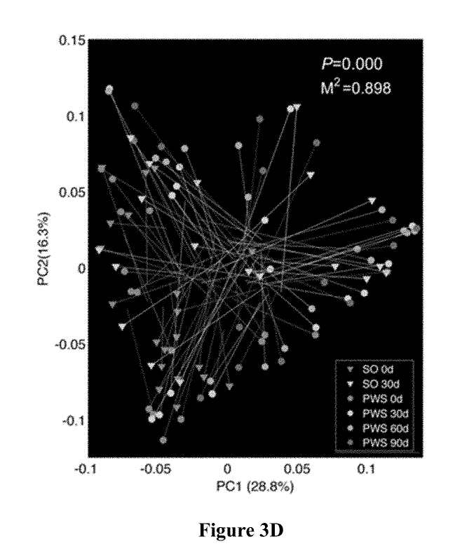

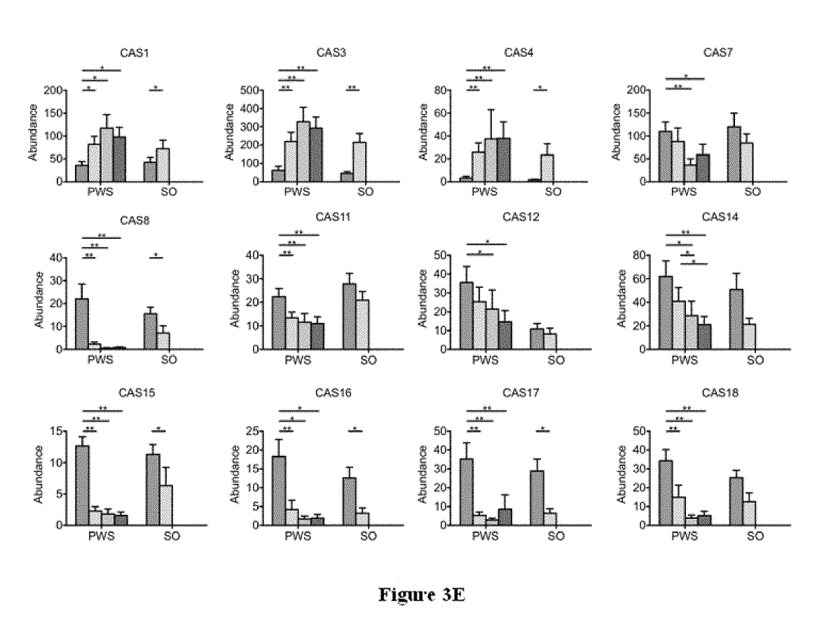

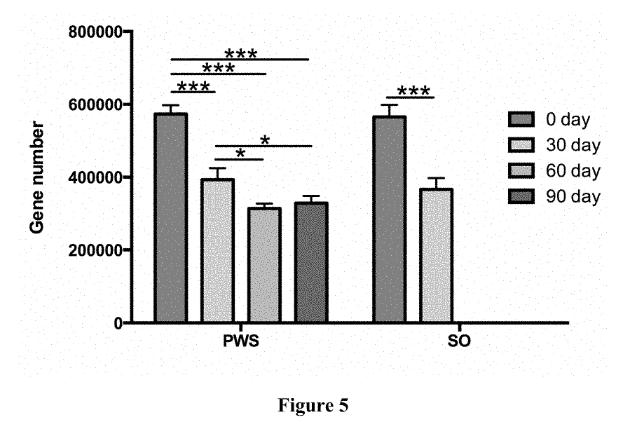

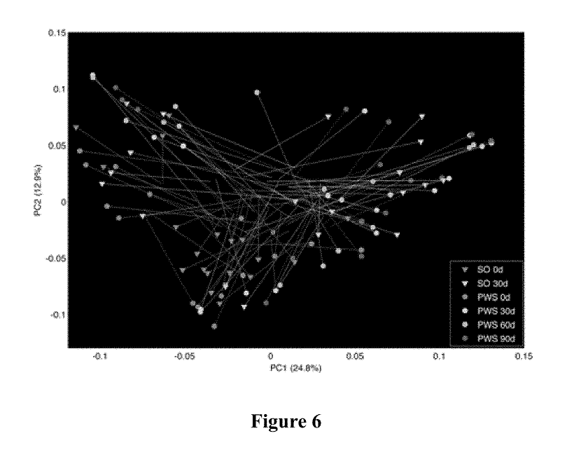

The composition of the gut microbiota showed a significant shift after 30 days of the intervention in both cohorts as indicated by principal coordinates analysis (PCoA, multivariate analysis of variance, (MANOVA) test, P=2.17e-6) based on Bray-Curtis dissimilarity of the 376 bacterial CAGs (FIGS. 3A and B). There was no significant difference in gut microbiota between PWS and SO both before (P=0.99) and after the intervention (P=0.8), suggesting that the PWS and SO gut microbiota were similarly dysbiotic prior to the intervention and that the intervention had the same effect on both (FIG. 3B). Analyses based on other β-diversity metrics and on pyrosequencing of the V1-V3 region of 16S rRNA genes confirmed similar finding (data not shown). On the other hand, the gene richness of the gut microbiota significantly reduced after the intervention (FIG. 5). More importantly, procrustes analysis combining PCoA of the 376 bacterial CAGs (FIG. 3A) with PCA of the bioclinical variables (data not shown) showed that the structural shifting of the gut microbiota based on the abundance of the bacterial CAGs was significantly associated with the changes of the bioclinical parameters of both PWS and SO cohorts, suggesting that the overall structural changes deep at the bacterial strain level were significantly associated with the improvements in host metabolic health (M²=0.891, Monte-Carlo P value<0.0001) (FIG. 6).

As species in other ecosystems such as rain forest, bacterial species in the human gut may also survive, adapt, and decline as functional groups responding to environmental perturbations (27-29). To identify species/strains in the gut ecosystem that responded as groups to the dietary intervention (30), we constructed a co-abundance network across all individuals and time points based on the 161 prevalent bacterial CAGs. Ward clustering algorithm and Permutational MANOVA (9999 permutations, P<0.001) based on bootstrapped Spearman correlation coefficients clustered these bacterial CAGs into 18 co-abundance species/strains (CAS) groups (FIG. 3C). Interestingly, different strains of the same species such as the 9Faecalibacterium prausnitzii genomes were clustered into different CAS groups, suggesting that different strains of the same species might occupy different metabolic niches in the gut ecosystem. Strains of the same species in the same CAS group were more similar in their genomic sequences to each other than strains of the same species clustered into different CASs, indicating that strains of the same species in different CAS groups may be functionally different (data not shown). Procrustes analysis showed that separations based on either CAS group abundance or host bioclinical variables before and after the intervention co-segregated along the first axis in both PWS and SO data sets, suggesting that the changes in the abundance of the various CASs were significantly associated with the improvements in host metabolic health (M²=0.898, Monte-Carlo P value<0.0001) (FIG. 3D). The agreement between strain-level and CAS-level procrustes analysis with host bioclinical variables (FIG. 3D) suggests that this strategy of organizing prevalent strains of human gut microbiota into co-abundance groups provides a potentially useful framework for understanding their functional interactions from each other and with the hosts.

Group level abundance analysis showed that 6 CASs, including CAS13 containing the most predominant species Prevotellacopri, did not change their abundance after the intervention (data not shown). CAS1, 3 and 4 significantly increased their abundance after the intervention while CAST, 8, 11, 12, 14, 15, 16, 17 and 18 decreased (FIG. 3E). CAS3 had a negative correlation with CAS8, 15, 16, and 18 (r>0.45, FDR<0.01) (FIG. 3C). CAS3 became the most enriched group after the dietary intervention. Notably, the major genomes in CAS3 were in the genus Bifidobacterium. Bifidobacteria utilize a wide range of carbohydrates, many of which are plant derived oligosaccharides and polysaccharides. The assembly for CAG00184, the most enriched genome after the intervention, covered 81.2% of the reference genome for Bifidobacterium pseudocatenulatum DSM 20438 with 98.6% identity (data not shown). The CAG00184 genome contained pathways for fermentation of monosaccharide, disaccharide, oligosaccharide and polysaccharide to produce acetate and lactate (data not shown). The large amount of non-digestible carbohydrates in the WTP diet therefore may have provided favorable nutritional conditions for proliferation of CAG00184. The carbohydrate-fermenting species such asB. pseudocatenulatum may work as “foundation species” to define much of the structure of a healthy gut ecosystem by rendering the gut environment unfavorable to pathogenic and detrimental bacteria, possibly via increased production of acetate (28, 31-33).

Isolation of B. pseudocatenulatum Guided by PCR-DGGE Technology from Post-Intervention Patients

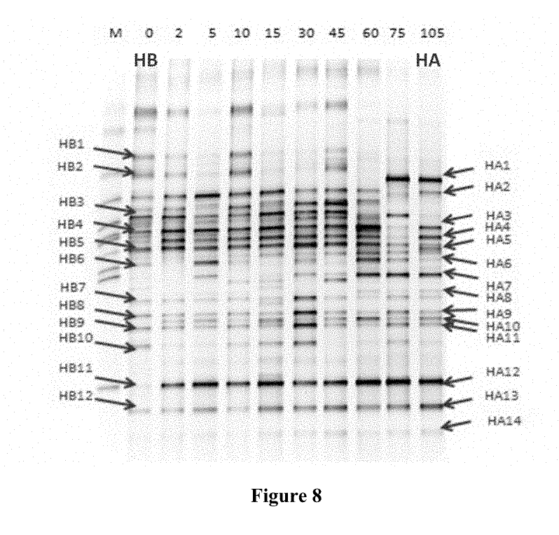

17 PWS obese children received a dietary intervention based on whole grains, traditional Chinese medicinal foods and prebiotics. During the dietary intervention, PWS children lost body weight and showed significant improvement of their metabolic health status such as fasting blood glucose and insulin. The composition of gut microbiota from the 17 PWS children was also significantly changed during intervention. Metagenomic analysis showed that Bifidobacterium spp. became the most promoted group after the dietary intervention, showing positive correlation to the improvement of various metabolic parameters. In this study, decrease of body weight and improvement of blood glucose and lipid profiles were observed in a PWS obese child (GD02) after 3-month dietary intervention. 16S rRNA V3 region PCR-DGGE fingerprinting of fecal bacteria from this PWS child on different time points during intervention was used to profile the compositional change of his gut microbiota. In FIG. 8, compared to pro-intervention, Band HAL Band HA7 and Band HA12 were significantly enriched along with intervention and became main bands at 105th day. Band HA12 had been one of the main bands since the next day after intervention (FIG. 8). Sequencing results indicated that the above 3 main bands were Lactobacillus spp. and Bifidobacterium spp. (Table 1). In conclusion, during dietary intervention, Lactobacillus spp. and Bifidobacterium spp. increased significantly and gradually became dominant bacteria in the gut of this PWS child. Co-abundance network based on metagenomic sequencing data showed that Bifidobacterium spp. negatively correlated with a lot of other species, suggesting Bifidobacterium spp. may be the key species contributing to host health improvement.

TABLE 1
Sequencing result of DGGE bands from fecal sample of a PWS volunteer
after dietary intervention for 105 days

| | | Similarity | | |
|---|---|---|---|---|
| DGGE band | Closest relatives | (%) | Phylum | Genus |
| HA1 | Lactobacillus | 100 | Firmicutes | Lactobacillus |
| | acidophilus 30SC | | | |
| | Sutterella stercoricanis | 89 | Proteobacteria | |
| | strain 5BAC4 | | | |
| HA7 | Lactobacillus mucosae | 100 | Firmicutes | Lactobacillus |
| | strain S32 | | | |
| HA12 | Bifidobacterium | 100 | Actinobacteria | Bifidobacterium |
| | pseudocatenulatum strain | | | |
| | B1279 | | | |

Isolation Method

0.6 g fecal sample from the PWS child at 105th days was mixed with 30 ml Ringer solution (0.1% L-Cysteine) in the anaerobic work station. The mixture was centrifuged for 5 min at 200 g. The supernatant was diluted from 10⁻¹ to 10⁻⁵. 200 μl of each dilution was spread on MRS Agar plates and incubated at 37° C. for 18 h in the anaerobic work station. 200 single colonies were selected randomly and the pure isolates were obtained by streaking into single colonies on plates.

16S rRNA V3 Region PCR-DGGE Profile of 168 Isolates and the Parental Fecal Sample.

The bands of 16S rRNA V3 region from 73 isolates were migrated to the identical position of Band HA12 in the original fecal sample, suggesting that we have isolated the Bifidobacterium spp. from the post-intervention fecal sample.

ERIC-PCR Classification of Bifidobacterium Spp. Isolates

According to the ERIC-PCR finger printing pattern, the 73 Bifidobacterium spp. isolates were classified into 5 different ERIC types. (Table 2).

TABLE 2
ERIC-PCR classification result of Bifidobacterium isolates and
Representative isolate of each ERIC type

| | DGGE | ERIC | Number | Representative |
|---|---|---|---|---|
| Corresponding Band | Types | Types | Of Isolates | Isolates |
| HA12 | IV | E7 | 55 | C95 |
| | | E8 | 14 | C1 |
| | | E9 | 1 | C55 |
| | | E10 | 1 | C62 |
| | | E11 | 1 | C15 |

16S rRNA 16S rRNA Gene Sequence Information

We queried the Genbank for closely related sequences of the 16S rRNA gene sequences of the represented isolates from 5 ERIC types. The nearest neighbor of the 5 represented isolates was Bifidobacterium pseudocatenulatum B1279 with higher than 99.6% homology.

TABLE 3
16S rRNA gene sequencing result of Bifidobacterium isolates representing each ERIC-
PCR type

| ERIC | DGGE | Number | Representative | Closest | Similarity with | |
|---|---|---|---|---|---|---|
| Types | Types | Of Isolates | Isolate | Neighbor | Closest Neighbor | Similarity with C95 |
| E7 | V | 55 | C95-1 | | 99.81 | 99.97 |
| | | | C95-3 | | 99.87 | 100.00 |
| | | | C95-5 | B1279 | 99.73 | 99.87 |
| | | | C95-7 | | 99.73 | 99.87 |
| | | | C95-14 | | 99.66 | 99.80 |
| E8 | V | 14 | C1-2 | | 99.80 | 99.87 |
| | | | C1-5 | B1279 | 99.87 | 100.00 |
| | | | C1-6 | | 99.87 | 100.00 |
| E9 | V | 1 | C55-1 | | 99.80 | 99.93 |
| | | | C55-3 | B1279 | 99.66 | 99.80 |
| | | | C55-5 | | 99.87 | 100.00 |
| E10 | V | 1 | C62-2 | | 99.66 | 99.80 |
| | | | C62-3 | B1279 | 99.73 | 99.87 |
| | | | C62-5 | | 99.87 | 99.93 |
| E11 | V | 1 | C15-2 | | 99.81 | 99.93 |
| | | | C15-3 | B1279 | 99.81 | 99.93 |
| | | | C15-5 | | 99.81 | 99.93 |

Whole Genome Sequence Information of Bifidobacterium pseudocatenulatum C95

Background:

21 SO (Simple Obesity) children accepted one-month dietary intervention in hospital. 17 PWS (Prader-Willi Syndrome) children accepted 3-month dietary intervention in hospital. We collected fecal samples of SO children at 0 day and 30th day. We also collected fecal samples of PWS children at the following time points: 0 day, 30th day, 60th day and 90th day. Total DNA was extracted from these fecal samples to carry out metagenomic sequencing. Through bioinformatics analysis we accomplished genome assembly of at single strain level and obtained 25 high-quality draft genome of Bifidobacterium pseudocatenulatum. Each child had its own draft genome with abundance information at different time points. Besides, we isolated a specific strain named B. pseudocatenulatum C95 from the fecal sample of GD02 child, and finished its whole genome sequence.

After comparing the high-quality B. pseudocatenulatum draft genome from GD02 with B. pseudocatenulatum C95 genome through MUMMER3.0, we found that their Identity and Query coverage were as follows: 99.93% and 99.39%, suggesting that this B. pseudocatenulatum draft genome was most probably B. pseudocatenulatum C95. The other 24 draft genomes also had high similarities with B. pseudocatenulatum C95, whose lowest Identity and Query coverage were at least as 98.63% and 86.26% respectively. (Note, the genome of B. pseudocatenulatum C95 is completed while the draft genomes of B. pseudocatenulatum were directly assembled from metagenomic sequences of fecal samples. There are regions, which could not be covered when the finished genome of B. pseudocatenulatum C95 was used as Reference genome, making the Reference coverage ranged from 80.75 to 88.54%.) Detailed alignments were listed in Table 4. Table 4 shows the alignment of 25 high quality draft genomes of B. pseudocatenulatum assembled from metagenomic datasets of fecal samples from 25 individuals with the finished genome of B. pseudocatenulatum C95 and their abundance changes during the intervention. It also shows that 23 of the 25 draft genomes of B. pseudocatenulatum increased their abundance after the intervention.

TABLE 4
Alignment of 25 high quality draft genomes of B. pseudocatenulatum with finished
genome of C95 and their abundance changes during intervention

| | | | | Identity | Abundance | Abundance | Abundance | Abundance | |
|---|---|---|---|---|---|---|---|---|---|
| ID | Reference | Ref_coverage | Query_coverage | (1-to-1) | (0 day) | (30 day) | (60 day) | (90 day) | Group |
| GD11 | C95 | 80.94 | 91.32 | 98.85 | 26.5 | 130 | | | S0 |
| GD13 | C95 | 83.05 | 86.26 | 98.7 | 14.4 | 5.08 | | | S0 |
| GD17 | C95 | 83.75 | 91.52 | 98.97 | 130 | 126.5 | | | S0 |
| GD20 | C95 | 82.59 | 91.21 | 98.99 | 0 | 73.5 | | | S0 |
| GD21 | C95 | 83.76 | 90.15 | 98.87 | 4.11 | 61.3 | | | S0 |
| GD23 | C95 | 87.02 | 97.2 | 99.79 | 3.96 | 84.8 | | | S0 |
| GD24 | C95 | 83.32 | 90.94 | 99.03 | 4.815 | 80.4 | | | S0 |
| GD26 | C95 | 82.63 | 94.07 | 98.92 | 0 | 14.1 | | | S0 |
| GD28 | C95 | 82.86 | 93.98 | 99.06 | 1 | 40 | | | S0 |
| GD29 | C95 | 82.29 | 93.92 | 99.04 | 45 | 105 | | | S0 |
| GD31 | C95 | 82.41 | 90.45 | 98.87 | 23.4 | 105 | | | S0 |
| GD32 | C95 | 80.75 | 90.05 | 98.63 | 8.83 | 38.1 | | | S0 |
| GD35 | C95 | 83.11 | 92.07 | 99.1 | 1.21 | 73 | | | S0 |
| GD02 | C95 | 88.54 | 99.39 | 99.93 | 14.9 | 22 | 343 | 188 | PWS |
| GD03 | C95 | 82.94 | 92.65 | 98.93 | 2.05 | 8.66 | 382 | 50.6 | PWS |
| GD04 | C95 | 81.47 | 92.09 | 98.99 | 2.16 | 4.66 | 130 | 27.7 | PWS |
| GD12 | C95 | 87.14 | 93.33 | 99.81 | 0.233 | 49.25 | 77.9 | 82.2 | PWS |
| GD15 | C95 | 83.54 | 91.64 | 98.97 | 1.79 | 130 | 57.6 | 120 | PWS |
| GD18 | C95 | 82.58 | 90.49 | 98.99 | 12.4 | 140 | 511 | 118 | PWS |
| GD41 | C95 | 83.97 | 88.32 | 98.96 | 0.567 | 14.2 | 24 | 69.7 | PWS |
| GD42 | C95 | 84.45 | 91.8 | 99.02 | 26.5 | 26.1 | 28.6 | 20.9 | PWS |
| GD43 | C95 | 81.05 | 90.53 | 98.87 | 1.08 | 21.3 | 34.6 | 3.345 | PWS |
| GD50 | C95 | 83.88 | 88.94 | 99 | 98.4 | 196 | 480.5 | 109 | PWS |
| GD52 | C95 | 82.53 | 91.44 | 98.89 | 0 | 3.67 | 6.4 | 14.3 | PWS |
| GD59 | C95 | 82.21 | 94.38 | 98.99 | 4.945 | 128 | 38.6 | 90.1 | PWS |
| CECT7765* | C95 | 86.68 | 85.08 | 98.62 | | | | | NA |

Notes:
CECT7765 information is based on information from US 20140369965.
ID: Individual id;
Reference: The genome used as reference genome in the genome comparison using MUMMER3.0;
Ref_coverage: The alignment coverage of reference genome;
Query_coverage: The alignment coverage of query genome, Identity (1-to-1): The percent identity (Number of alignment blocks comprising the 1-to-1 mapping of reference to query. This is a subset of the M-to-M mapping, with repeats removed);
SO: Simple obesity children who received the hospitalized intervention for 30 days, thus having the abundance on 0 day and 30 day;
PWS: PWS (Prader-Willi Syndrome) children who received the hospitalized intervention for 90 days, thus having the abundance on 0 day, 30 day, 60 day and 90 day).

B. pseudocatenulatum C95 has a completed (finished) genome. Compared with the C95 completed genome, B. pseudocatenulatum B1279 has 98.16% identity with B. pseudocatenulatum C95 completed genome and C95 covered 86.3% of B1279.

### Establishment of Foundation Species Reduced Metabolic Deteriorations

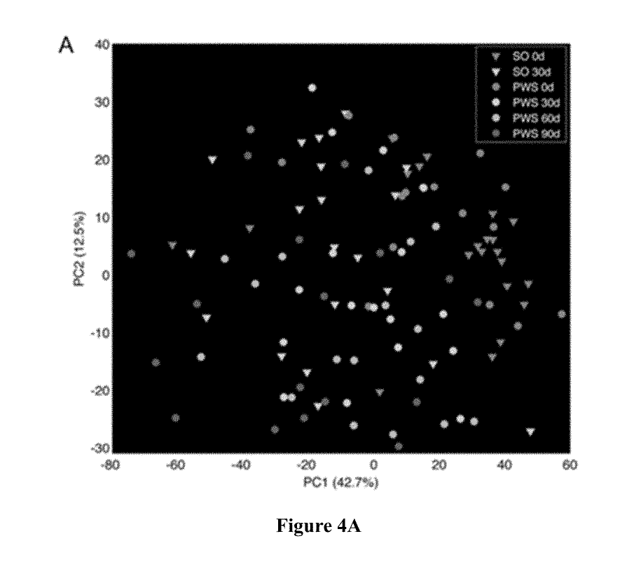

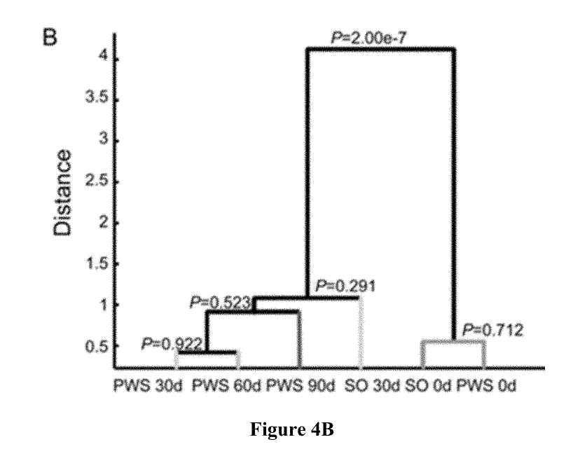

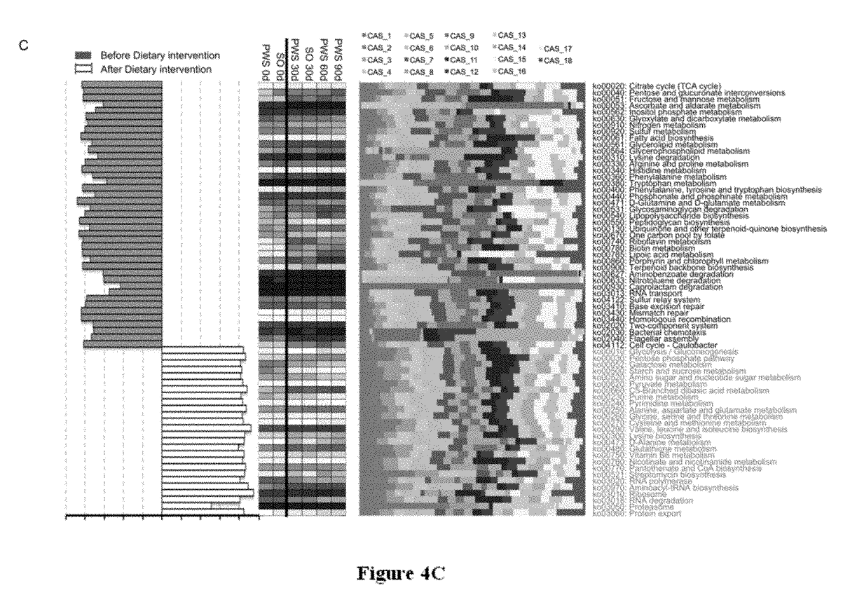

To see how the altered population structure of the gut microbiota affected its metabolic potential, we profiled the metagenomic data using HUMAnN to identify and quantify genes within metabolic pathways (34). In total, 5,234 KEGG orthology groups (KOs) were recognized and quantified. The PCA score plot of all the KOs showed a significant shift after the intervention (MANOVA test, P=2.00e-7, FIGS. 4A and B), indicating a modulation of the metabolic capacity of the gut microbiota concomitant with its diet-induced structural changes. There was no significant difference between the PWS and SO cohorts either before or after the intervention (MANOVA P=0.712 and P=0.291, FIG. 4B). Thus, gut microbiota between PWS and SO children shared similar structural and functional features both before and after the intervention.

Using the linear discriminant analysis (LDA) effect size (LEfSe) method (35), 67 KEGG database metabolic pathways (P<0.05) were identified as significantly responding to the dietary intervention (data not shown). 41 of these pathways were significantly decreased and 26 were enriched after the intervention. Notable among the enriched pathways were those for carbohydrate catabolism, including starch and sucrose metabolism (ko00500), and amino sugar and nucleotide sugar metabolism (ko00520). Notable among the decreased pathways were those for fat and protein metabolism, including fatty acid biosynthesis (ko00061), phenylalanine metabolism (ko00360), and tryptophan metabolism (ko00380). In addition, lipopolysaccharide biosynthesis (ko00540), peptidoglycan biosynthesis (ko00550) and flagellar assembly (ko02040) pathways were decreased, suggesting reduced bacterial antigen synthesis after the intervention. Pathways for xenobiotics biodegradation (ko00627, ko00633 and ko00930), and DNA repair-related pathways (ko03410, ko03430 and ko03440) were also decreased, perhaps reflecting reduced toxin load and mutagenic stress in the gut microbiota environment after the intervention.

Thus, the metabolic potential of the post-intervention gut microbiota, as determined by its genetic composition was significantly changed, in agreement with a reduced capacity in inducing metabolic deteriorations as shown by the gut microbiota transplantation test.

### Establishment of Foundation Species Changed Gut Microbiota to a Healthier Structure

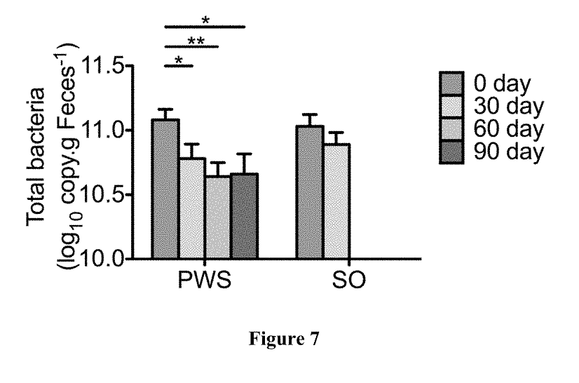

The interventional diet had dramatically increased non-digestible carbohydrates, which may get into the colon to potentially shift the fermentation metabolism of the gut microbiota. Score plots of PCA and orthogonal projection to latent structure-discriminant analysis (OPLS-DA) of NMR-based metabonomic profiling data of fecal water samples from the SO (Day 0 and 30) and the PWS cohorts (Day 0, 30, 60 and 90) showed significant shift of metabolite composition after the intervention (data not shown). OPLS-DA coefficient plots showed dramatic increase of non-digestible carbohydrates after the intervention (data not shown). Nineteen fecal metabolites in SO cohort and 18 in PWS were found to be significantly reduced by the intervention (data not shown). Among these significantly reduced metabolites many were bacterial products. A significant decrease of these bacterial metabolites in the gut was concomitant with a significant reduction in the total gut bacterial load as determined by qPCR (FIG. 7). Despite the decrease of bacterial metabolites, the relative concentration of acetate, a beneficial metabolite (36, 37), was increased among short chain fatty acids (SCFAs) while those of isobutyrate and isovalerate were decreased (data not shown). Acetate is produced from carbohydrate fermentation while isobutyrate and isovalerate are produced from amino acid fermentation (38, 39). Trimethylamine (TMA), a toxic metabolite produced when gut bacteria ferment choline derived from dietary fats (40), was decreased in fecal water after the intervention (data not shown). Thus, metabolic profiling of fecal water indicates a shift from fat and protein fermentation to carbohydrate fermentation in the gut after the intervention, in agreement with the identified changes of KEGG pathways (FIG. 4C). The cytotoxicity of fecal water samples to cultured human Caco-2 cells was significantly reduced in both SO and PWS cohorts after the intervention, indicating that the post-intervention microbiota may have produced less toxic metabolites in the gut (data not shown).

To more closely examine how the dietary intervention changed the carbohydrate metabolism of the gut microbiota, we searched all the 2,077,766 non-redundant genes against a downloaded dbCAN database to identify carbohydrate-active enzyme (CAZy) genes (31, 41). 84,549 genes were assigned to 299 CAZy families. The PCA score plot of the 299 families significantly separated the pre- and post-intervention samples, indicating a significant shift in the genes for carbohydrate metabolism in the gut microbiome (data not shown). Genes for degradation of starch, inulin and cellulose were significantly enriched, while genes for degradation of glycosylated compounds of animal origin such as mucin were significantly depleted in the microbiome after the intervention (data not shown) (41). Genes for formate-tetrahydrofolate ligase participating in acetate production (17, 29), were significantly increased after the intervention, being consistent with the increased relative concentration of acetate among the fecal SCFAs (data not shown). These shifts reflect the increased availability of plant carbohydrates in the colon, favoring proliferation of bacteria such as bifidobacteria that contain carbohydrate-fermenting genes and produce beneficial metabolites such as acetate (39). A recent metagenomic study of gut microbiota in colon cancer patients also found increased protein and fat fermentation and decreased carbohydrate fermentation over healthy controls (42), indicating that shifting gut microbiota metabolism with increased carbohydrates in the gut may help alleviate metabolic deteriorations of a diverse range of chronic diseases.

Taken together, metagenomic analysis of the gut microbiota and metabolite profiling of fecal water samples indicate that the dietary intervention shifted the gut microbiota in both cohorts to a healthier structure dominated by carbohydrate-fermenting bacteria with significantly reduced production of toxic metabolites, regardless of the genetic background of the cohorts. In other words, the establishment of foundation species changed gut microbiota to a healthier structure dominated by carbohydrate-fermenting bacteria with significantly reduced production of toxic metabolites.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

### REFERENCE CITED

14. S. Xiao et al., A gut microbiota-targeted dietary intervention for amelioration of chronic inflammation underlying metabolic syndrome. FEMS Microbiol Ecol 87, 357 (February, 2014).
15. N. Fei, L. Zhao, An opportunistic pathogen isolated from the gut of an obese human causes obesity in germfree mice. ISME J 7, 880 (April, 2013).
17. P. J. Turnbaugh et al., An obesity-associated gut microbiome with increased capacity for energy harvest. Nature 444, 1027 (Dec. 21, 2006).
18. S. T. Papavramidis, E. V. Kotidis, O. Gamvros, Prader-Willi syndrome-associated obesity treated by biliopancreatic diversion with duodenal switch. Case report and literature review. J Pediatr Surg 41, 1153 (June, 2006).
19. G. Labruna et al., High leptin/adiponectin ratio and serum triglycerides are associated with an “at-risk” phenotype in young severely obese patients. Obesity 19, 1492 (July, 2011).
20. J. Zweigner, R. R. Schumann, J. R. Weber, The role of lipopolysaccharide-binding protein in modulating the innate immune response. Microbes Infect 8, 946 (March, 2006).
21. P. D. Cani et al., Metabolic endotoxemia initiates obesity and insulin resistance. Diabetes 56, 1761 (July, 2007).
22. R. E. Ley et al., Obesity alters gut microbial ecology. Proc Natl Acad Sci USA 102, 11070 (Aug. 2, 2005).
23. P. J. Turnbaugh et al., A core gut microbiome in obese and lean twins. Nature 457, 480 (Jan. 22, 2009).
24. A. Cotillard et al., Dietary intervention impact on gut microbial gene richness. Nature 500, 585 (Aug. 29, 2013).
25. E. Le Chatelier et al., Richness of human gut microbiome correlates with metabolic markers. Nature 500, 541 (Aug. 29, 2013).
26. H. B. Nielsen et al., Identification and assembly of genomes and genetic elements in complex metagenomic samples without using reference genomes. Nature biotechnology 32, 822 (August, 2014).
27. Y. Zhou et al., Biogeography of the ecosystems of the healthy human body. Genome Biol 14, R1 (Jan. 14, 2013).
28. A. M. Ellison et al., Loss of foundation species: consequences for the structure and dynamics of forested ecosystems. Frontiers in Ecology and the Environment 3, 479 (November, 2005).
29. M. J. Claesson et al., Gut microbiota composition correlates with diet and health in the elderly. Nature 488, 178 (Aug. 9, 2012).
30. L. A. David et al., Diet rapidly and reproducibly alters the human gut microbiome. Nature 505, 559 (Jan. 23, 2014).
31 P. Scott, S. W. Gratz, P. O. Sheridan, H. J. Flint, S. H. Duncan, The influence of diet on the gut microbiota. Pharmacol Res 69, 52 (March, 2013).
32. S. Fukuda et al., Bifidobacteria can protect from enteropathogenic infection through production of acetate. Nature 469, 543 (Jan. 27, 2011).
33. G. R. Gibson, X. Wang, Regulatory effects of bifidobacteria on the growth of other colonic bacteria. J Appl Bacteriol 77, 412 (October, 1994).
34. S. Abubucker et al., Metabolic reconstruction for metagenomic data and its application to the human microbiome. PLoS Comput Biol 8, e10023 58 (2012).
35. N. Segata et al., Metagenomic biomarker discovery and explanation. Genome Biol 12, R60 (2011).
36. K. M. Maslowski et al., Regulation of inflammatory responses by gut microbiota and chemoattractant receptor GPR43. Nature 461, 1282 (Oct. 29, 2009).
37. V. Tremaroli, F. Backhed, Functional interactions between the gut microbiota and host metabolism. Nature 489, 242 (Sep. 13, 2012).
38. W. R. Russell et al., High-protein, reduced-carbohydrate weight-loss diets promote metabolite profiles likely to be detrimental to colonic health. American Journal of Clinical Nutrition 93, 1062 (May, 2011).
39. H. J. Flint, K. P. Scott, S. H. Duncan, P. Louis, E. Forano, Microbial degradation of complex carbohydrates in the gut. Gut Microbes 3, 289 (July-August, 2012).
40. Z. Wang et al., Gut flora metabolism of phosphatidylcholine promotes cardiovascular disease. Nature 472, 57 (Apr. 7, 2011).
41. B. L. Cantarel, V. Lombard, B. Henrissat, Complex carbohydrate utilization by the healthy human microbiome. PLoS One 7, e28742 (2012).
42. G. Zeller et al., Potential of fecal microbiota for early-stage detection of colorectal cancer. Mol Syst Biol 10, 766 (2014).

1. A composition comprising:
(1) a Bifidobactgerium pseudocatenulatem strain C95 with accession No. CGMCC10549, wherein the genome of the C95 strain is designated as a reference genome;
(2) a highly similar strain, wherein the highly similar strain comprises a genome that is designated as a query genome, wherein when aligned,
the query genome covers at least 86% of the reference genome,
the query and reference genomes share at least 98.7% sequence identity in aligned regions; or
(3) a strain derived therefrom; and
(4) a pharmaceutically acceptable or dietary carrier.

2. The composition of claim 1, wherein the reference genome further covers at least 87% of the query genome.

3. The composition of claim 1, wherein the query genome covers at least 93% of the reference genome, the reference genome further covers at least 87% of the query genome, and the query and reference genomes share at least 98.7% sequence identity in aligned regions.

4. The composition of claim 1, which comprises strain C95.

5. The composition of claim 1, wherein the composition is a pharmaceutical composition.

6. The composition of claim 1, wherein the composition is a nutritional supplement or a nutritive composition.

7. The composition of claim 1, wherein the composition comprises at least 103 to 1014 colony forming units of strain C95 or the highly similar strain per gram or millimeter of the composition.

8. The composition according to claim 5, further comprising a Lactobacillus mucosae strain.

9. The composition of claim 1, wherein the composition comprises cell components, metabolites, secreted molecules, or any combinations thereof, of strain C95 or the highly similarly strain.

10. A method for preparing a composition of claim 1, comprising formulating the Bifidobactgerium pseudocatenulatem strain C95 or the highly similar strain into a suitable composition.

11. The method of claim 10, wherein the composition comprises strain C95 and a Lactobacillus mucosae strain.

12. A method for the prevention and/or treatment of a disease selected from the group consisting of overweight, obesity, hyperglycemia, diabetes, fatty liver, dyslipidemia, metabolic syndrome, infections in obese or overweight subjects and/or adipocyte hypertrophy, simple or genetic obesity, metabolic deteriorations and inflammation, said method comprising the administration of a composition of claim 1 to a subject in need thereof.

13. The method of claim 12, wherein the composition comprises strain C95 and a Lactobacillus mucosae strain.

14. (canceled)

15. (canceled)

16. A method for establishing as foundation species that define the structure of a healthy gut ecosystem, rendering a gut environment unfavorable to pathogenic and detrimental bacteria, reducing the concentration of enterobacteria in intestinal content with respect to an untreated control, the method comprising the administration of a composition of claim 1 to a subject in need thereof.

17. The method of claim 16, wherein the composition comprises strain C95 and a Lactobacillus mucosae strain.

18. (canceled)

19. The method of claim 12, wherein the diabetes is type II diabetes.

20. (canceled)

21. The method of claim 12, wherein the subject suffers from Prader-Willi Syndrome.
